SEQUENCE LISTING (4 sequences)

SEQ ID NO: 1 - protein, 252 aa, Homo sapiens
MYRMQLLSCIALSLALVTNSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 20 aa, Homo sapiens
MYRMQLLSCIALSLALVTNS

SEQ ID NO: 3 - protein, 109 aa, Homo sapiens
PELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 4 - protein, 104 aa, Homo sapiens
QPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

(19) United States
(12) Patent Application Publication
(10) Pub. No.: US 2025/0176833 A1
(43) Pub. Date: Jun. 5, 2025

(54) FC DOMAIN IMAGING PROBES AND METHODS OF USE THEREOF

(71) Applicant: University of Saskatchewan, Saskatoon (CA)

(72) Inventors: Clarence Ronald Geyer, Saskatoon (CA); Wendy Lynn Bernhard, Saskatoon (CA)

(21) Appl. No.: 18/691,390

(22) PCT Filed: Sep. 12, 2022
(86) PCT No.: PCT/CA2022/051359

§ 371 Date: Mar. 12, 2024

Related U.S. Application Data
(60) Provisional application No. 63/243,411, filed on Sep. 13, 2021.

(51) Int. Cl. A61B 5/00; A61B 10/00; A61K 49/00

(52) U.S. Cl. CPC A61B 5/0071; A61B 10/0041; A61K 49/0032; A61K 49/0058

(57) ABSTRACT

The disclosure relates to a method of detecting the presence of tumor infiltrating Fc receptor-expressing immune cells in a mammalian subject afflicted with a cancer or detecting the presence of one or more site(s) of inflammation characterized by Fc receptor-expressing immune cells in a mammalian subject afflicted with an inflammatory disease or disorder, the method comprising administering a non-specific imaging probe comprising one or more Fc domain(s) labeled with a detectable moiety to the subject; subjecting the subject to imaging; and identifying one or more tumor(s) or sites of inflammation comprising infiltrating Fc receptor-expressing immune cells.

### CROSS-REFERENCE TO RELATED APPLICATIONS

This application claims the benefit of priority to U.S. Provisional Application No. 63/243,411, filed Sep. 13, 2021, the contents of which is incorporated herein by reference in its entirety.

### INCORPORATION OF SEQUENCE LISTING

A computer readable form of the Sequence Listing “13764-P64723US01_Sequence_Listing” (4,984 bytes) created on Jan. 28, 2025, is herein incorporated by reference.

### FIELD

The present disclosure relates to non-specific imaging probes comprising fragment crystallizable (Fc) domain(s) and their use in the molecular imaging of immune cells in cancers and inflammatory diseases or disorders.

### BACKGROUND

Tracking the immune response is important but complex due to the mixture of cell types, variability in cell populations, and dynamic environment. Tissue biopsies and blood analysis can be used to identify infiltrating and circulating immune cells, however due to the dynamic nature of the immune response these are prone to sampling errors.

The innate immune system is the first to respond to infection or injury with recruitment of granulocytes and then monocytes [1]. This sets off a cascade of signals amplifying the acute inflammatory response. Since the response is self-limiting, inflammation is usually resolved after a few days, however, if factors that mediate resolution are not in place, chronic inflammation can lead to inflammatory diseases such as Crohn's disease, psoriasis, type II diabetes, atherosclerosis, and cancer [1]. Cancers develop from transformed cells that have managed to evade the immune system and gain growth advantage over normal cells [2]. In doing this, cancer cells also manage to use immune cells such as monocytes and neutrophils to aid in their development [3]. Having a way to identify the innate immune cell recruitment will be useful for disease diagnosis and cancer treatment.

Current techniques used to monitor immune cells are invasive and prone to error.

In solid tumors, a biopsy is collected, and histopathology is used to determine immune cell infiltration [4, 5]. This is equivalent to taking a ‘snapshot’ of the tumor, which is dynamic and can change over time.

Molecular imaging agents have been developed for noninvasive molecular imaging and show promise [7-12]. [18F]-(fluorodeoxyglucose) ([18F]-FDG) for example is the gold standard molecular imaging probe currently used to image cancer and has also been used to image immune responses using PET [6]. [18F]-FDG is taken up by most tumors but is also taken up by myeloid cells. This can make it difficult to distinguish between inflammation in the tumor and cancer cells in some cases [13-15]. Some cancer therapies specifically those targeting the immune response or ionizing radiation can increase tumor infiltration by immune cells [16, 17]. Follow-up [18F]-FDG imaging that show an increase in uptake may actually be detecting inflammation and not tumor growth [6, 15]. Imaging probes are needed that directly image immune cells and are able to distinguish between tumor cells and inflammation.

### SUMMARY

Noninvasive molecular imaging of immune cell infiltration allows for multiple imaging time points to be obtained after imaging probe injection if necessary [6]. In research, this is useful since it cuts down on the number of animals required for an experiment. In the clinic, this can lead to identification of immune infiltration and provides a way to monitor therapeutic efficacy. Non-invasive, targeted molecular imaging provides a method to monitor immune response, which has advantages of providing whole-body images, being non-invasive, and allowing longitudinal monitoring.

In the present disclosure, three non-specific Fc domain-containing proteins (Fc, scFv-Fc, and IgG) were labeled with the near infrared dye IRDye800CW and used as imaging probes to assess tumor infiltrating immune cells in FaDu and A-431 xenograft models. It was shown that non-specific Fc domains localize to tumors and are visible by fluorescent imaging. This tumor localization correlates with binding to immune cells with some xenografts showing higher fluorescent signals than others. The Fc domain alone bound to different human immune cell types. The Fc domain can be a valuable tool to study innate immune response and to image tumor infiltrating immune cells of patients.

Accordingly, a first aspect of the disclosure includes a non-specific imaging probe comprising one or more Fc domain(s) labeled with a detectable moiety, wherein the non-specific imaging probe is for use in detecting tumor infiltrating Fc receptor-expressing immune cells in a mammalian subject afflicted with a cancer.

A second aspect of the disclosure includes a non-specific imaging probe comprising one or more Fc domain(s) labeled with a detectable moiety, wherein the non-specific imaging probe is for use in detecting one or more site(s) of inflammation characterized by Fc receptor-expressing immune cells in a mammalian subject afflicted with an inflammatory disease or disorder.

Another aspect of the disclosure includes use of a non-specific imaging probe comprising one or more Fc domain(s) labeled with a detectable moiety for detecting tumor infiltrating Fc receptor-expressing immune cells in a mammalian subject afflicted with a cancer or one or more site(s) of inflammation characterized by Fc receptor-expressing immune cells in a mammalian subject afflicted with an inflammatory disease or disorder.

Another aspect of the disclosure includes a method of detecting the presence of tumor infiltrating Fc receptor-expressing immune cells in a mammalian subject afflicted with a cancer or detecting one or more site(s) of inflammation characterized by Fc receptor-expressing immune cells in a mammalian subject afflicted with an inflammatory disease or disorder, the method comprising:
a. administering a non-specific imaging probe comprising one or more Fc domain(s) labeled with a detectable moiety to the subject;
b. subjecting the subject to imaging; and
c. identifying the presence of Fc receptor-expressing immune cells,
wherein detection of the detectable moiety is indicative of the presence of Fc receptor-expressing immune cells.

Another aspect of the disclosure includes a method of detecting the presence of tumor infiltrating Fc receptor-expressing immune cells in a mammalian subject afflicted with a cancer, the method comprising:
a. administering a non-specific imaging probe comprising one or more Fc domain(s) labeled with a detectable moiety to the subject;
b. subjecting the subject to imaging; and
c. identifying one or more tumor(s) comprising infiltrating Fc receptor-expressing immune cells,
wherein detection of Fc receptor-expressing immune cells is indicative of the presence of infiltrating immune cells in the one or more tumor(s).

Another aspect of the disclosure includes a method of detecting the presence of one or more site(s) of inflammation characterized by Fc receptor-expressing immune cells in a mammalian subject afflicted with an inflammatory disease or disorder, the method comprising:
a. administering a non-specific imaging probe comprising one or more Fc domain(s) labeled with a detectable moiety to the subject;
b. subjecting the subject to imaging; and
c. identifying one or more site(s) of inflammation,
wherein detection of an accumulation of Fc receptor-expressing immune cells is indicative of the presence of one or more site(s) of inflammation.

A further aspect of the disclosure includes a method of identifying a subject afflicted with a cancer as having a poor prognosis, the method comprising
a. detecting the presence of tumor infiltrating Fc receptor-expressing immune cells in the subject using a method disclosed herein; and
b. quantifying the level of neutrophils and/or macrophages in one or more tumor(s); c. quantifying the level of neutrophils and/or macrophages in a normal surrounding tissue sample; and
d. comparing the level of neutrophils and/or macrophages in the one or more tumor(s) with the normal surrounding tissue sample,
wherein an increased level of neutrophils and/or macrophages in the one or more tumor(s) as compared to the level of neutrophils and/or macrophages in the normal surrounding tissue sample is indicative of a poor prognosis.

### BRIEF DESCRIPTION OF THE DRAWINGS

Embodiments of the present disclosure will now be described in relation to the drawings in which:

FIG. 1 depicts an image of the proteins used in this study, which are a control IgG (˜150 kDa), a control scFv-Fc (˜110 kDa), a targeted VHH-Fc (˜80 kDa) and a Fc (˜50 kDa).

FIG. 2A depicts a graph illustrating VHH-Fc (targeted) and control scFv-Fc tested for binding and saturation on FaDu cells by titration. FIG. 2B depicts optical imaging of the targeted VHH-Fc (top) and control scFv-Fc (bottom) imaging in CD-1 nude mice bearing FaDu xenografts over time. FIG. 2C depicts a graph illustrating the normalized fluorescence of the mouse imaging shown in FIG. 2B. FIG. 2D depicts a graph illustrating the tumor-to-background (TBR) of the mouse imaging shown in FIG. 2B. Error bars shown are representative of standard error of mean from at least three independent mice. * is p-value≤0.05. X is xenograft.

FIG. 3A depicts graphs illustrating targeted VHH-Fc and Fc tested for binding and saturation on MDA-MB-231 cells by titration. FIG. 3B (top) depicts optical imaging of the targeted VHH-Fc in CD-1 nude mice bearing MDA-MB-231 xenografts over time. FIG. 3B (bottom) depicts optical imaging of CD-1 nude mice bearing MDA-MB-231 xenografts injected with 75× unlabeled Fc followed by IRDye800CW-labeled VHH-Fc. FIG. 3C depicts a graph illustrating normalized fluorescence of mouse imaging shown in FIG. 3B. Error bars shown are representative of standard error of mean from at least three independent mice and *** is p-value≤0.001 and ***p-value is ≤0.0001.

FIG. 4A depicts optical imaging of control IgG, control scFv-Fc and Fc injected into CD-1 nude mice bearing FaDu and imaged over time, the 24 hour image is shown. FIG. 4B depicts optical imaging of control IgG, control scFv-Fc and Fc injected into CD-1 nude mice bearing A431 and imaged over time, the 24 hours image is shown. FIGS. 4C and 4D depicts a graph illustrating normalized fluorescence of mouse imaging over time. FIGS. 4E and 4F depict graphs illustrating tumor-to-background ratio (TBR) of mouse imaging over time. Error bars shown are representative of standard error of mean from at least three independent mice and p-value * is p-value≤0.05, ** is p-value≤0.01, *** is p-value≤0.001 and *** is p-value≤ 0.0001

FIG. 5A depicts dot plot of representative flow cytometry with lineage marker and IRDye800CW-Fc. FIG. 5B depicts a graph illustrating the percent of positive cells for Fc and Fc+lineage marker in the xenografts. FIG. 5C depicts a graph illustrating percent of positive cells for Fc and Fc+lineage marker in the bone marrow.

FIG. 6 depicts graphs illustrating PBMCs incubated with the IRDye680RD-Fc and an immune panel then analyzed by flow cytometry to determine the type of cells it binds.

FIG. 7 depicts a series of graphs that illustrate Fc binding to different cell lines using flow cytometry. HL-60 is a myeloblast cell line that differentiates into granulocytes. Mv4-11 is a macrophage cell line. Jurkat is a T-cell line. K562 are hematopoietic but spontaneously differentiate into erythrocytes, granulocyte and monocytes. SEM is a B-cell line.

FIG. 8A depicts a graph illustrating % purity of the proteins labeled with IRDye680RD or IRDye800CW and expressed in expi293 cells and purified with a MabSelect SuRe purification column. FIG. 8B depicts a graph illustrating the molecular weight of the proteins labeled with IRDye680RD or IRDye800CW and expressed in expi293 cells and purified with a MabSelect SuRe purification column.

FIG. 9 depicts two graphs illustrating liver-to-background and kidney-to-background ratios for VHH-Fc and scFv-Fc for CD-1 nude mice bearing FaDu xenografts. The line is drawn at a TBR of 1. Error bars show the standard error of mean and representative of at least three mice.

FIG. 10 depict graphs illustrating the flow cytometry results of control IgG, scFv-Fc and Fc1 mixed FaDu or A-431 cells.

FIG. 11 depicts a series of graphs illustrating liver-to-background and kidney-to-background ratios for IgG, scFv-Fc and Fc in CD-1 nude mice imaged over time. Error bars show the standard error of mean and representative of at least three mice.

### DETAILED DESCRIPTION

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g., Green, M. and Sambrook, J. (2012) Molecular Cloning: A Laboratory Manual. 4th Edition, Vol. II, Cold Spring Harbor Laboratory Press, New York).

Thus, for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the term “non-specific imaging probe” and variations thereof, refers to an imaging probe comprising an Fc domain and lacking a targeting moiety or any domain, portion or region that is capable of targeting to a particular cell type as compared to the non-specific binding of the Fc domain on its own. For example, the imaging probe does not comprise an antigen binding fragment or other targeting moiety which targets the imaging probe to a particular cell type but may be linked/conjugated/attached to a non-specific molecule, for example an antigen binding fragment, that is not expected to bind to the cells with which the imaging probe is intended to bind.

As used herein “antigen binding fragment” refers to for example a Fab, Fab′, F(ab′)2, scFv, dsFv, ds-scFv, dimer, minibody, diabody, or multimer thereof or bispecific antibody fragment, or a combination thereof.

As used herein, the term “targeting moiety” and variations thereof, refers to a molecule that specifically binds a target cell. “Specifically binding” refers to having greater affinity to a particular cell type of interest relative to alternative cell types.

As used herein, the term “tumor infiltrating Fc receptor-expressing immune cell” and variations thereof, refer to one or more Fc receptor-expressing immune cells in a subject that have migrated to one or more tumors in a subject afflicted with a cancer. Examples of tumor infiltrating Fc receptor-expressing immune cells include natural killer cells, natural killer T-cells (NK-T) cells, dendritic cells, macrophages, neutrophils, eosinophils, and mast cells.

As used herein, the term “detectable moiety” and variations thereof, optionally a label, refers to an atom, molecule or composition, wherein the presence of the atom, molecule or composition can be directly or indirectly measured. Detectable moieties include, for example, chemiluminescent moieties, bioluminescent moieties, fluorescent moieties, microbubbles, MRI probes, radionuclides, radioisotopes, and metals. Examples of radioisotopes include 89Z, 68Ga, and 99mTc.

As used herein, the term “imaging” and variations thereof refers to methods for detecting detectable moieties. Such a detectable moiety/label can be detected, for example, by fluorescence imaging, by visual inspection, by fluorescence spectroscopy, by reflectance measurement, by flow cytometry, by X-rays, by a variety of fluorescent endoscopy methods (including but not limited to laparoscopy, gastroscopy, bronchoscopy, cystoscopy, ureteroscopy, arthroscopy and colonoscopy), by positron emission tomography (PET), single photon emission computed tomography (SPECT), ultrasound, by computed tomography (CT), by a variety of magnetic resonance methods such as magnetic resonance imaging (MRI) and magnetic resonance spectroscopy (MRS). Different types of imaging may be combined, for example fluorescent imaging can be used to achieve 3D imaging when combined with other modalities such as CT or MRI.

As used herein, the term “Fc domain” and variations thereof, refers to the fragment crystallizable domain of an antibody and typically comprises at least the last two constant domains of each heavy chain of an antibody which dimerize to form the Fc domain. The structure of the Fc domain of an antibody is dependent on the isotype of the antibody. For example, the Fc domain of an IgG1 antibody comprises the CH2 and CH3 domains.

As used herein, the term “tumor” and variations thereof refers to all neoplastic masses of tissue, and all pre-cancerous and cancerous tissue growths, including solid tumors.

The term “cancer” and variations thereof refer to any malignant and/or invasive proliferation, growth or tumor caused by abnormal cell growth. As used herein “cancer” includes solid tumors named for the type of cells that form them. The term “cancer” includes, but is not limited to, a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one.

As used herein, the term “site of inflammation” refers to an area of tissue in which Fc receptor-expressing immune cells have accumulated and are present in larger quantities than in surrounding normal tissue.

The term “afflicted” includes subjects suffering from the disease and/or diagnosed with the disease. For example, a subject afflicted with cancer includes mammalian subjects that have developed cancer naturally (for example without external human intervention) or as a result of external human intervention (for example a mouse implanted with cancerous xenografts).

As used herein, the term “poor prognosis” and variations thereof, refers to low rates of survival, low responsiveness or non-responsiveness to treatments, for example via the treatment being inhibited, optionally by immune cells, and/or susceptibility to adverse reactions from treatments.

As used herein, the term “inflammatory disease or disorder” refers to diseases or disorders characterized by inflammation where the inflammation is not caused by infection. Examples of such disease are well known in the art and include Crohn's disease, atherosclerosis, inflammatory bowel disorder, arthritis, colitis, type II diabetes, cancer, or psoriasis.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Accordingly, an aspect of the disclosure includes a non-specific imaging probe comprising one or more Fc domain(s) labeled with a detectable moiety, wherein the non-specific imaging probe is for use in detecting tumor infiltrating Fc receptor-expressing immune cells in a mammalian subject afflicted with a cancer.

Another aspect of the disclosure includes a non-specific imaging probe comprising one or more Fc domain(s) labeled with a detectable moiety, wherein the non-specific imaging probe is for use in detecting sites of inflammation characterized by Fc receptor-expressing immune cells in a mammalian subject afflicted with an inflammatory disease or disorder.

In some embodiments, the non-specific imaging probe may be linked/conjugated/attached to a non-specific molecule, for example an antigen binding fragment, optionally a scFv, that is not expected to bind to the cells with which the imaging probe is intended to bind. For example, it could be a benefit to have a non-specific scFv conjugated to the Fc domain of a non-specific imaging probe as it would increase circulation time allowing it more time to bind its target.

In some embodiments, the non-specific imaging probe comprises one or more Fc domain(s) which are not conjugated/attached to any non-specific molecule. For example, using a Fc domain that is not conjugated to a non-specific molecule in imaging has a shorter circulation and penetration/uptake time due to its size compared to a larger protein. In imaging this is beneficial because a lower background signal compared to the targeted tissue will result sooner and because it clears from the body faster, potentially producing less side-affects since exposure is for a lesser duration (for example, this could mean less radiation exposure if it is attached to a radioactive moiety). The Fc domain alone also binds slightly stronger than molecules with extra domains, for example IgG, which can cause steric hinderance. Additionally, conjugating an Fc domain to a non-specific molecule, for example an scFv, could lead to off target binding or effects. Additionally, non-specific imaging probes comprising Fc domains with no conjugation to a non-specific molecule are easier to synthesize, purify and have better stability.

In one embodiment, the one or more Fc domain(s) is/are one or more IgG1 Fc domain(s). In some embodiments, the one or more Fc domain(s) is/are IgG2, IgG3, or IgG4 or a combination thereof. In some embodiments, the one or more Fc domain(s) comprise(s) a signal sequence and CH2 and CH3 domains. In some embodiments, the CH2 and CH3 domains are from an IgG2, IgG3, or IgG4 or a combination thereof. In an embodiment, the one or more Fc domain(s) is/are from a non-human species that has an Fc domain that will bind human Fc receptors. In some embodiments, the one or more Fc domain(s) is/are one or more mammalian Fc domain(s). In some embodiments, the one or more Fc domain(s) is/are one or more mouse Fc domain(s). In some embodiments, the signal sequence is an interleukin 2 (IL2) signal sequence. In other embodiments, the signal sequence is selected from serum albumin, CD5, trypsinogen, prolactin and/or Immunoglobulin Kappa light chain signal sequences. In some embodiments, the one or more Fc domain(s) is/are one or more human Fc domain(s). In another embodiment, the one or more Fc domain(s) comprise(s) the amino acid sequence set forth in SEQ ID NO: 1. In some embodiments, the one or more Fc domain(s) comprise(s) an amino acid sequence that has at least about 85%, at least about 90%, at least about 95%, at least about 96%, at least about 97%, at least about 98%, or at least about 99% sequence identity to SEQ ID NO:1.

In some embodiments, the detectable moiety is a fluorescent moiety. Probes comprising fluorescent moieties may be useful for image-guided surgery or if the disease is close to the surface. In another embodiment, the fluorescent moiety is a near infrared (NIR) dye. In further embodiments, the detectable moiety is IRDye680RD or IRDye800CW, optionally IRDye680RD NHS ester or IRDye800CW NHS ester. In another embodiment, the detectable moiety is a radioisotope, optionally indium-111, or metals. In another embodiment, the detectable moiety is a PET or SPECT radionuclide, a fluorescent molecule, or a microbubble.

In one embodiment, the non-specific imaging probe comprises one Fc domain. In other embodiments, the non-specific imaging probe comprises a plurality of Fc domains. In some embodiments, the non-specific imaging probe comprises at least 2 Fc domains. In another embodiment, the non-specific imaging probe comprises 1 to 10 Fc domains, optionally 1 Fc domain, 2 Fc domains, 3 Fc domains, 4 Fc domains, 5 Fc domains, 6 Fc domains, 7 Fc domains, 8 Fc domains, 9 Fc domains, or 10 Fc domains. In some embodiments, the at least 2 Fc domains are joined or linked together. Fc domains can be joined or linked together using one of a variety of methods known to a person skilled in the art. For example, Fc domains can be joined or linked together using click chemistry, optionally using a dibenzocyclooctyne group (DBCO) and azide moiety, optionally wherein each of the Fc domains being joined or linked together comprise either an azide moiety or DBCO group and an Fc domain comprising an azide moiety is linked to another Fc domain comprising a DBCO group via a reaction, optional under physiological conditions.

In some embodiments, the Fc receptor-expressing immune cells comprise natural killer cells, NK-T cells, dendritic cells, macrophages, neutrophils, eosinophils, and/or mast cells. In another embodiment, the Fc receptor-expressing immune cells are neutrophils or macrophages.

In one embodiment, the tumor is a solid tumor. In another embodiment, the tumor is a liquid tumor.

In one embodiment, the mammalian subject is a mouse that has developed cancer, optionally naturally or as a result of human intervention, for example via implantation of cancerous xenografts. In another embodiment the mammalian subject is a human. In another embodiment, the mammalian subject is a cat, dog, hamster, or livestock such as a cow, pig, or sheep.

The cancer may be any cancer. In some embodiments, the cancer is any cancer that is characterized by solid tumors. In another embodiment, the cancer is breast cancer, hepatocellular carcinoma, squamous cell carcinoma, esophageal squamous cell carcinoma, melanoma, colorectal cancer, pulmonary adenocarcinoma, or renal cell carcinoma. In one embodiment, the cancer is breast cancer. In another embodiment, the cancer is squamous cell carcinoma. In some embodiments, the cancer is any cancer that is characterized by liquid tumors, for example a liquid tumor of the bone marrow or spleen.

In one embodiment, the subject is afflicted with a cancer and the non-specific imaging probe is for administration to or for use in the subject for the identification of one or more tumor(s) comprising tumor infiltrating Fc receptor-expressing immune cells, wherein detection of the Fc receptor-expressing immune cells is indicative of the presence of tumor infiltrating Fc receptor-expressing immune cells in the one or more tumor(s).

In another embodiment, the subject is afflicted with an inflammatory disease or disorder and the non-specific imaging probe is for use in the subject for the identification of one or more sites of inflammation, wherein detection of the Fc receptor-expressing immune cells is indicative of the presence of site of inflammation. In some embodiments, the inflammatory disease or disorder is Crohn's disease, atherosclerosis, inflammatory bowel disorder, arthritis, colitis, type II diabetes or psoriasis. In one embodiment, the non-specific imaging probe is for use in the subject prior to receiving a treatment for an inflammatory disease or disorder. In another embodiment, the non-specific imaging probe is for use in the subject after receiving a treatment for an inflammatory disease or disorder. In a further embodiment, the non-specific imaging probe is for use in the subject prior to and after receiving a treatment for an inflammatory disease or disorder.

In some embodiments, the non-specific imaging probe is for administration to or for use in the subject more than once at different time periods, for example to monitor response to treatments. In some embodiments, the non-specific imaging probe is for administration to or for use in the subject at least twice within a week, a month or within a year. In some embodiments, the non-specific imaging probe is for administration or use once every week, once every month, or once every year. In other embodiments, the non-specific imaging probe is for administration to or for use in the subject every month, every 2 months, every 3 months, every 4 months, every 5 months, every 6 months, every 7 months, every 8 months, every 9 months, every 10 months, or every 11 months. In some embodiments, the non-specific imaging probe is for administration to or for use in the subject at diagnosis, of optionally cancer, and optionally after treatment one or more times.

In an embodiment, the non-specific imaging probe is for administration to or for use in the subject prior to receiving a cancer treatment. In another embodiment, the non-specific imaging probe is for administration to or for use in the subject after receiving a cancer treatment. In a further embodiment the non-specific imaging probe is for administration to or for use in the subject prior to receiving a cancer treatment and after receiving a cancer treatment. In some embodiments, the non-specific imaging probe is for administration or use to monitor response to therapies and/or altering a therapy, for example, a particular treatment may be administered or used, maintained, altered, stopped or switched in response to the detection of tumor infiltrating Fc receptor-expressing immune cells.

In some embodiments, the non-specific imaging probe is formulated for injection, optionally intramuscular injection, or an intranasal formulation, intravenous formulation, oral formulation, inhalation formulation or direct injection into the tumor. The imaging probe may be formulated in a similar formulation as any immunotherapy, formulations for which are well known in the art, for example the formulation may comprise PBS and a preservative. In some embodiments, the non-specific imaging probe is for administration or use via injection. In some embodiments, the non-specific imaging probe is formulated for intramuscular injection, intranasal, intravenous, oral, or inhalatory administration. In some embodiments, the non-specific imaging probe for administration or use is a composition comprising the non-specific imaging probe and a pharmaceutically acceptable excipient, diluent, or one or more other additive(s). In some embodiments, the composition comprises PBS and/or a preservative.

In some embodiments, the non-specific imaging probe is formulated for administration or use in a vector comprising a polynucleotide encoding the amino acid sequence of SEQ ID NO: 1 or an amino acid sequence having at least about 85%, at least about 90%, or at least about 95% sequence identity to SEQ ID NO: 1.

Another aspect of the disclosure includes a method of detecting the presence of tumor infiltrating Fc receptor-expressing immune cells in a mammalian subject afflicted with a cancer, the method comprising:
a. administering a non-specific imaging probe comprising one or more Fc domain(s) labeled with a detectable moiety to the subject;
b. subjecting the subject to imaging; and
c. identifying one or more tumor(s) comprising infiltrating Fc receptor-expressing immune cells,
   wherein detection of Fc receptor-expressing immune cells is indicative of the presence of infiltrating immune cells in the one or more tumor(s).

In some embodiments, the non-specific imaging probe is any one of the non-specific imaging probes described herein. In some embodiments the method of detection is used for monitoring immune response in a therapeutic animal model, optionally a mouse model, wherein the animal is afflicted with cancer.

In some embodiments, the subject is afflicted with a cancer. In another embodiment, the subjecting the subject to imaging comprises imaging one or more tumor(s), and the presence of Fc receptor-expressing immune cells is indicative of the presence of one or more tumor(s) comprising infiltrating Fc receptor-expressing immune cells.

Another aspect of the disclosure includes a method of detecting the presence of one or more site(s) of inflammation characterized by Fc receptor-expressing immune cells in a mammalian subject afflicted with an inflammatory disease or disorder, the method comprising:
a. administering a non-specific imaging probe comprising one or more Fc domain(s) labeled with a detectable moiety to the subject;
b. subjecting the subject to imaging; and
c. identifying one or more site(s) of inflammation,
   wherein detection of an accumulation of Fc receptor-expressing immune cells is indicative of the presence of one or more site(s) of inflammation.

In another embodiment, the subject is afflicted with an inflammatory disease or disorder and the presence of Fc receptor-expressing immune cells is indicative of one or more site(s) of inflammation. In another embodiment, the inflammatory disease or disorder is Crohn's disease, atherosclerosis, inflammatory bowel disorder, arthritis, colitis, type II diabetes or psoriasis. In another embodiment, the non-specific imaging probe is for use or administration in the subject prior to receiving a treatment for an inflammatory disease or disorder. In another embodiment, the non-specific imaging probe is for use or administration in the subject after receiving a treatment for an inflammatory disease or disorder. In another embodiment, the non-specific imaging probe is for use or for administration in the subject prior to and after receiving a treatment for an inflammatory disease or disorder.

In one embodiment, the one or more Fc domain(s) is/are one or more IgG1 Fc domain(s). In some embodiments, the one or more Fc domain(s) is/are IgG2, IgG3, or IgG4 or a combination thereof. In some embodiments, the one or more Fc domain(s) comprise(s) an IL2 signal sequence and CH2 and CH3 domains. In some embodiments, the CH2 and CH3 domains are from an IgG2, IgG3, or IgG4 or a combination thereof. In some embodiments, the one or more Fc domain(s) is/are one or more human Fc domain(s). In another embodiment, the one or more Fc domain(s) comprise(s) the amino acid sequence set forth in SEQ ID NO: 1. In some embodiments, the one or more Fc domain(s) comprise(s) an amino acid sequence that has at least about 70%, at least about 75%, at least about 80%, at least about 85%, at least about 90%, at least about 96%, at least about 97%, at least about 98%, or at least about 99% sequence identity to SEQ ID NO: 1.

In some embodiments, the detectable moiety is a fluorescent moiety. In another embodiment, the fluorescent moiety is a near infrared (NIR) dye. In further embodiments, the detectable moiety is IRDye680RD or IRDye800CW, optionally IRDye680RD NHS ester or IRDye800CW NHS ester. In another embodiment, the detectable moiety is a radioisotope, optionally indium-111, or a metal. In another embodiment, the detectable moiety is a PET or SPECT radionuclide, a fluorescent molecule, or a microbubble.

In one embodiment, the non-specific imaging probe comprises one Fc domain. In other embodiments, the non-specific imaging probe comprises a plurality of Fc domains. In some embodiments, the non-specific imaging probe comprises at least 2 Fc domains. In another embodiment, the non-specific imaging probe comprises 1 to 10 Fc domains, optionally 1 Fc domain, 2 Fc domains, 3 Fc domains, 4 Fc domains, 5 Fc domains, 6 Fc domains, 7 Fc domains, 8 Fc domains, 9 Fc domains, or 10 Fc domains.

In some embodiments, the Fc receptor-expressing immune cells comprise natural killer cells, NK-T cells, dendritic cells, macrophages, neutrophils, eosinophils, and/or mast cells. In another embodiment, the Fc receptor-expressing immune cells are neutrophils or macrophages.

In one embodiment, the tumor is a solid tumor.

In one embodiment, the mammalian subject is a mouse that has developed cancer, optionally naturally or as a result of human intervention, for example via implantation of cancerous xenografts. In another embodiment the mammalian subject is a human.

In some embodiments, the cancer is any cancer that is characterized by solid tumors. In another embodiment, the cancer is breast cancer, hepatocellular carcinoma, squamous cell carcinoma, esophageal squamous cell carcinoma, melanoma, colorectal cancer, pulmonary adenocarcinoma, or renal cell carcinoma. In one embodiment, the cancer is breast cancer. In another embodiment, the cancer is squamous cell carcinoma.

In some embodiments, the subject is imaged more than once at different time periods, for example to monitor response to treatments. In some embodiments, the subject is imaged at least twice within a week, within a month or within a year. In some embodiments, the subject is imaged once every week, once every month, or once every year. In other embodiments, the subject is imaged every month, every 2 months, every 3 months, every 4 months, every 5 months, every 6 months, every 7 months, every 8 months, every 9 months, every 10 months, or every 11 months. In some embodiments, the subject is imaged every 24 hours, every 48 hours, every 72 hours, every 96 hours, every 120 hours, or every 144 hours.

In an embodiment, the subject is imaged prior to receiving a cancer treatment. In another embodiment, the subject is imaged after receiving a cancer treatment. In a further embodiment the subject is imaged prior to receiving a cancer treatment and after receiving a cancer treatment. In some embodiments, the subject is imaged to monitor response to therapies and/or altering a therapy, for example, a particular treatment may be administered or used, maintained, altered, stopped or switched in response to the detection of tumor infiltrating Fc receptor-expressing immune cells.

In some embodiments, the non-specific imaging probe is formulated for injection, optionally intramuscular injection, or an intranasal formulation, intravenous formulation, oral formulation, or inhalation formulation. In some embodiments, the non-specific imaging probe is for administration or use via injection. In some embodiments, the non-specific imaging probe is formulated for intramuscular injection, intranasal, intravenous, oral, or inhalatory administration. In some embodiments, the non-specific imaging probe for administration or use is a composition comprising the non-specific imaging probe and a pharmaceutically acceptable carrier, excipient, diluent, or one or more other additive(s).

In some embodiments, the non-specific imaging probe is formulated for administration or use in a vector comprising a polynucleotide encoding the amino acid sequence of SEQ ID NO: 1 or an amino acid sequence having at least about 70%, at least about 75%, at least about 80%, at least about 85%, at least about 90%, or at least about 95% sequence identity to SEQ ID NO: 1.

A further aspect of the disclosure includes a method of identifying a mammalian subject afflicted with a cancer as having a poor prognosis, the method comprising
a. detecting the presence of tumor infiltrating Fc receptor-expressing immune cells in the subject using a method of detection described herein;
b. quantifying the level of neutrophils and/or macrophages in one or more tumor(s);
c. quantifying the level of neutrophils and/or macrophages in a normal surrounding tissue sample; and
d. comparing the level of neutrophils and/or macrophages in the one or more tumor(s) with the normal surrounding tissue sample,
   wherein an increased level of neutrophils and/or macrophages in the one or more tumor(s) as compared to the level of neutrophils and/or macrophages in the normal surrounding tissue sample is indicative of a poor prognosis.

In some embodiments, the method further comprises isolating the one or more tumor(s) or a biopsy of the one or more tumor(s). In some embodiments, the type(s) of Fc receptor-expressing immune cells is/are detected by subjecting the one or more tumor(s) or the biopsy of the one or more tumor(s) to flow cytometry or immunohistochemistry.

In some embodiments, the method of identifying the subject as having a poor prognosis is for use in monitoring the immune response in a therapeutic animal model, optionally in a mouse model.

In some embodiments, the method of identifying the subject as having a poor prognosis further comprises the step of quantifying the Fc receptor-expressing immune cells, optionally neutrophils or macrophages, detected. In some embodiments, an increased number of neutrophils in diseased tissue such as a tumor as compared to normal surrounding tissue, optionally wherein the cancer is hepatocellular carcinoma, esophageal squamous cell carcinoma, melanoma, colorectal cancer, pulmonary adenocarcinoma, or renal cell carcinoma, is indicative of a poor prognosis. In some embodiments, the quantification of the number of Fc receptor-expressing immune cells comprises imaging diseased tissue such as a tumor biopsy or tumor. In some embodiments, the quantification of the number of Fc receptor-expressing immune cells comprises comparing the amount of Fc receptor-expressing immune cells, optionally neutrophils or macrophages, in diseased tissue, such as a tumor biopsy or tumor, versus normal tissue using for example, imaging or immunohistochemistry.

In some embodiments, the methods described herein are for use in monitoring response to cancer therapies and/or altering a cancer therapy, for example, a particular treatment may be administered or used, maintained, altered, stopped or switched in response to the detection of tumor infiltrating Fc receptor-expressing immune cells.

Another aspect of the disclosure includes use of a non-specific imaging probe comprising one or more Fc domain(s) labeled with a detectable moiety for detecting tumor infiltrating Fc receptor-expressing immune cells in a mammalian subject afflicted with a cancer or for detecting one or more site(s) of inflammation characterized by Fc receptor-expressing immune cells in a mammalian subject afflicted with an inflammatory disease or disorder. In some embodiments, the non-specific imaging probe is any one of the non-specific imaging probes described herein. In some embodiments the method of detection is used for monitoring immune response in a therapeutic animal model, optionally a mouse model, wherein the animal is afflicted with cancer.

In some embodiments, the subject is afflicted with a cancer and the non-specific imaging probe is for use in the subject for the identification of one or more tumor(s) comprising infiltrating Fc receptor-expressing immune cells, wherein detection of the Fc receptor-expressing immune cells is indicative of the presence of infiltrating Fc receptor-expressing immune cells in the one or more tumor(s).

In another embodiment, the subject is afflicted with an inflammatory disease or disorder and the non-specific imaging probe is for use in the subject for the identification of one or more site(s) of inflammation, wherein detection of the Fc receptor-expressing immune cells is indicative of the presence of one or more site(s) of inflammation. In some embodiments, the inflammatory disease or disorder is Crohn's disease, atherosclerosis, inflammatory bowel disorder, arthritis, colitis, type II diabetes or psoriasis. In one embodiment, the non-specific imaging probe is for use in the subject prior to receiving a treatment for an inflammatory disease or disorder. In another embodiment, the non-specific imaging probe is for use in the subject after receiving a treatment for an inflammatory disease or disorder. In a further embodiment, the non-specific imaging probe is for use in the subject prior to and after receiving a treatment for an inflammatory disease or disorder.

In one embodiment, the one or more Fc domain(s) is/are one or more IgG1 Fc domain(s). In some embodiments, the one or more Fc domain(s) is/are IgG2, IgG3, or IgG4 or a combination thereof. In some embodiments, the one or more Fc domain(s) comprise(s) an IL2 signal sequence and CH2 and CH3 domains. In some embodiments, the CH2 and CH3 domains are from an IgG2, IgG3, or IgG4 or a combination thereof. In some embodiments, the one or more Fc domain(s) is/are one or more human Fc domain(s). In another embodiment, the one or more Fc domain(s) comprise(s) the amino acid sequence set forth in SEQ ID NO: 1. In some embodiments, the one or more Fc domain(s) comprise(s) an amino acid sequence that has at least about 70%, at least about 75%, at least about 80%, at least about 85%, at least about 90%, at least about 96%, at least about 97%, at least about 98%, or at least about 99% sequence identity to SEQ ID NO: 1.

In some embodiments, the detectable moiety is a fluorescent moiety. In another embodiment, the fluorescent moiety is a near infrared (NIR) dye. In further embodiments, the detectable moiety is IRDye680RD or IRDye800CW, optionally IRDye680RD NHS ester or IRDye800CW NHS ester. In another embodiment, the detectable moiety is a radioisotope, optionally indium-111, or other metals. In another embodiment, the detectable moiety is a PET or SPECT radionuclide, a fluorescent molecule, or a microbubble.

In one embodiment, the non-specific imaging probe comprises one Fc domain. In other embodiments, the imaging probe comprises a plurality of Fc domains. In some embodiments, the non-specific imaging probe comprises at least 2 Fc domains. In another embodiment, the non-specific imaging probe comprises 1 to 10 Fc domains, optionally 1 Fc domain, 2 Fc domains, 3 Fc domains, 4 Fc domains, 5 Fc domains, 6 Fc domains, 7 Fc domains, 8 Fc domains, 9 Fc domains, or 10 Fc domains.

In some embodiments, the Fc receptor-expressing immune cells comprise natural killer cells, NK-T cells, dendritic cells, macrophages, neutrophils, eosinophils, and/or mast cells. In another embodiment, the Fc receptor-expressing immune cells are neutrophils or macrophages.

In one embodiment, the tumor is a solid tumor.

In one embodiment, the mammalian subject is a mouse that has developed cancer, optionally naturally or as a result of human intervention, for example via implantation of cancerous xenografts. In another embodiment the mammalian subject is a human.

In some embodiments, the cancer is any cancer that is characterized by solid tumors. In another embodiment, the cancer is breast cancer, hepatocellular carcinoma, squamous cell carcinoma, esophageal squamous cell carcinoma, melanoma, colorectal cancer, pulmonary adenocarcinoma, or renal cell carcinoma. In one embodiment, the cancer is breast cancer. In another embodiment, the cancer is squamous cell carcinoma.

In some embodiments, the subject is imaged more than once at different time periods, for example to monitor response to treatments. In some embodiments, the subject is imaged at least twice within a week, within a month or within a year. In some embodiments, the subject is imaged once every week, once every month, or once every year. In other embodiments, the subject is imaged every month, every 2 months, every 3 months, every 4 months, every 5 months, every 6 months, every 7 months, every 8 months, every 9 months, every 10 months, or every 11 months. In some embodiments, the subject is imaged every 24 hours, every 48 hours, every 72 hours, every 96 hours, every 120 hours, or every 144 hours.

In an embodiment, the subject is imaged prior to receiving a cancer treatment. In another embodiment, the subject is imaged after receiving a cancer treatment. In a further embodiment the subject is imaged prior to receiving a cancer treatment and after receiving a cancer treatment. In some embodiments, the subject is imaged to monitor response to therapies and/or altering a therapy, for example, a particular treatment may be administered or used, maintained, altered, stopped or switched in response to the detection of tumor infiltrating Fc receptor-expressing immune cells.

In some embodiments, the non-specific imaging probe is formulated for injection, optionally intramuscular injection, or is an intranasal formulation, intravenous formulation, oral formulation, or inhalation formulation. In some embodiments, the non-specific imaging probe is for administration or use via injection. In some embodiments, the non-specific imaging probe is formulated for intramuscular injection, intranasal, intravenous, oral, or inhalatory administration. In some embodiments, the non-specific imaging probe for administration or use is a composition comprising the non-specific imaging probe and a pharmaceutically acceptable carrier, excipient, diluent, or one or more other additive(s).

In some embodiments, the non-specific imaging probe is formulated for administration or use in a vector comprising a polynucleotide encoding the amino acid sequence of SEQ ID NO: 1 or an amino acid sequence having at least about 70%, at least about 75%, at least about 80%, at least about 85%, at least about 90%, or at least about 95% sequence identity to SEQ ID NO: 1.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

The immune system protects the body from foreign molecules and helps regulate overgrowth of misregulated cells. There are many different types of cells involved in the innate and acquired immune response. Non-invasively monitoring immune response provides tools to identify infections, tumors, and monitor efficacy and safety of therapies.

In this study, imaging properties of Fc containing proteins (IgG, scFv-Fc and Fc) in mouse xenografts were examined. Both the single chain variable fragment (scFv-Fc) and Fc domain accumulated in mouse xenografts, where the scFv-Fc accumulated at higher levels relative to the Fc domain. Therefore, attaching an Fc domain to an existing targeting moiety was also tested along with Fc domains.

### Methods

### Cloning VHH-Fc, Fc Receptor, and Expression

The human Fc domain was cloned using the pFUSEss-CHIg-hG1 vector (Invivogen) by removing DNA sequence after the IL2 signal sequence (including the CH1 domain) up to the CH2 domain. The resulting Fc domain contains the IL2 signal sequence and CH2 and CH3 domains, which gives the following protein sequence: MYRMQLLSCIALSLALVTNSEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNG KEYKCKVSNKALPAPIEKTISKAKGQPREPQVYTLPPSREEMTKNQVSLTCLVKGFYPSDIA VEWESNGQPENNYKTTPPVLDSDGSFFLYSKLTVDKSRWQQGNVFSCSVMHEALHNHYT QKSLSLSPGK (SEQ ID NO: 1). Within SEQ ID NO:1, the sequence MYRMQLLSCIALSLALVTNS (SEQ ID NO: 2) is the IL2 signal sequence, the sequence PELLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAK (SEQ ID NO: 3) is the CH2 and the sequence QPREPQVYTLPPSREEMTKNQVSLTCLVKGFYPSDIAVEWESNGQPENNYKTTPPVLDSD GSFFLYSKLTVDKSRWQQGNVFSCSVMHEALHNHYTQKSLSLSP (SEQ ID NO: 4) is the CH3 sequence. The VHH-Fc targets FaDu and MDA-MB-231 cells and contains a VHH linked to the Fc described above. The control IgG and scFv-Fc were sub-cloned from a control antigen binding fragment (Fab) as previously described [18, 19].

For protein expression, 1 μg of total DNA from the control IgG, control scFv-Fc, VHH-Fc, or Fc expressing plasmids per milliliter of cells were transfected into Expi293 cells (ThermoFisher) and expressed according to the manufacturer's instructions. Briefly, enhancers were added for one week. Cells and supernatant were separated by centrifugation at 1000×g for 5 minutes. The supernatant was collected and filtered through a 0.45 μm filter unit (Millipore). Antibodies and fragments were purified from the supernatant using an AKTAprime plus (GE Healthcare Life Sciences) with a HiTrap MabSelect SuRe column (GE Healthcare Life Sciences), according to the manufacturer's instructions. The purified protein was sterilized using a 2 μm filter unit and dialyzed with 1 liter of PBS two times using a 7 MWCO dialysis tubing. 2.5 μg of purified protein was analyzed by microcapillary electrophoresis using an Agilent Bioanalyzer 2100, according to the manufacturer's instructions.

### Tissue Culture

Expi293 cell line was purchased from Thermofisher Scientific. SEM cells were purchased from (DSMZ). All other cell lines were purchased from ATCC. Mv4-11, SEM, and K562 cells were cultured in 90% Iscove's Modified Dulbecco's Medium (IMDM) and 10% fetal bovine serum (FBS). Jurkat, A-431, and DLD-1 cells were cultured in 90% Roswell Park Memorial Institute medium (RPMI) and 10% FBS. FaDu cells were cultured in 90% Minimum Essential Medium (MEM/EBSS) with Earle's balanced salts and 10% FBS. MDA-MB-231 cells were cultured in 90% Dulbecco Modified Eagle Medium (DMEM) and 10% FBS.

### Protein Labeling and Flow Cytometry

IgG, scFv-Fc, VHH-Fc, or Fc (1 mg) were labeled with IRDye680RD NHS ester (LI-COR Biosciences) for flow cytometry or IRDye800CW NHS ester (LI-COR Biosciences) for optical imaging experiments, according to the manufacturer's instructions. Briefly, the Fc, IgG, VHH-Fc, and Fc were labeled at a 1:3 protein:dye ratio in PBS at 4° C. overnight on a rotator. Excess dye was removed using a Zeba spin desalting column (7 MWCO), according to the manufacturer's instructions (Thermofisher Scientific).

Flow cytometry was performed on cancer cell lines mv4-11 (macrophage Jurkat (T-cell), SEM (B-cell), K562 (hematopoietic), HL-60 (promyeloblast), A-431 (epithelial), FaDu (epithelial), and MDA-MB-231 (epithelial). 1×10⁵ cells were collected for each titration point. Cells were washed with PBSF (phosphate buffered saline and 2% fetal bovine serum) and suspended in PBSF. IRDye680RD-labeled proteins (0-2 μM) were titrated with 1×10⁵ cells, incubated at room temperature for 30 minutes, and then cooled on ice for 15 minutes. Cells were washed with PBSF and analyzed on a Beckman Coulter Gallios flow cytometer. The data was analyzed using FlowJo version 10.5.3 and Graphpad Prism version 6. Mean fluorescent intensity (MFI) values were normalized and analyzed by a non-linear regression curve fit to obtain KD values. For single point binding analysis, the unstained population was set to 0.5% in a FL7 (IRDye680RD) positive gate. The % positive cells in that gate at 100 nM of IRDye680RD-labeled protein were recorded.

Flow cytometry experiments with human peripheral blood were performed by lysing the red cells with red cell lysis buffer (150 mM NH4Cl, 10 mM NaHCO3, 1.3 mM EDTA) for 10 minutes at room temperature. Cells were centrifuged at 1,000 rpm for 5 minutes, washed with PBSF, and counted. 3×10⁶ cells were stained with immune panel antibodies (CD3, CD4, CD19, CD14, CD8, CD56, CD16, CD7, CD45) with or without IRDye680RD-Fc for 20 minutes at room temperature. Cells were washed and analyzed on a Beckman Coulter Gallios flow cytometer.

The data was analyzed using Kaluza Flow cytometry analysis software. Cells were gated on singlets plotting forward scatter (FSC) vs FSC and on intact cells by plotting FSC vs side scatter (SS). Using the intact gate, white blood cells (WBC) were gated by plotting CD45 vs SS. The lymphocyte population was further refined using the WBC gate and plotting FS vs SS. Lymphocytes and myelocytes/monocytes were separated using the WBC gate and plotting CD45 vs SS. T-cells and B-cells were separated by using the lymphocyte gate and plotting CD3 vs CD19. The myelocyte/monocytes gate was separated into monocytes and granulocytes by plotting CD14 vs CD16. IRDye680RD-Fc that bound to B-cells were analyzed using the B-cell gate and plotting CD19 vs Fc-680. IRDye680RD-Fc that bound to T-cells were analyzed using the T-cell gate and plotting CD3 vs Fc-680. IRDye680RD-Fc that bound to monocytes were analyzed using the monocyte gate and plotting CD14 vs Fc-680. IRDye680RD-Fc that bound to granulocytes were analyzed using the granulocyte gate and plotting CD16 vs Fc-680.

### Mouse Xenograft Models, Imaging, and Xenograft and Bone Marrow Flow Cytometry

All mouse experiments were performed with approval and under the supervision and guidelines of the University of Saskatchewan Animal Care Committee. Female CD-1 nude mice were obtained from Charles River Canada. All mice had ad libitum access to food and water. Mice 6-8 weeks old were used for experiments. For xenografts, ten million cells (FaDu, A-431, or MDA-MB-231) were washed with serum free media and suspended in 50 μL of serum free media and 50 μL of Matrigel Matrix (Corning) on ice. The cell mixture was injected subcutaneously into the right hind flank of CD-1 nude female mice. Xenografts were monitored with external calipers until they reached a size of 150-300 mm3. Once xenografts reached this size the mice were injected with 0.5 nmoles of IRDye800CW-labeled IgG, scFv-Fc, VHH-Fc, or Fc. The mice were imaged over time using a LI-COR Pearl small animal imaging system. For the Fc blocking experiment 75×(37.5 nmoles) the amount of unlabeled Fc was injected prior to the injection of the VHH-Fc.

Three regions of interest (ROI) were selected from equal sized areas containing the same number of pixels for xenografts, liver, kidney, and contralateral side. The MFI of ROIs were averaged then normalized by labeling ratio. A two-way analysis of variance (ANOVA) with multiple comparisons using GraphPad Prism was used to compare the normalized fluorescent signals and tumor-to-background ratios (TBR) for accumulation of the different proteins and blocking experiment in xenografts. All experiments were performed with at least three biological replicates (n≥3) and represent the standard error of the mean (SEM).

Flow cytometry from mouse xenografts and bone marrow were performed in mice bearing A-431 and FaDu xenografts 24 hours after injection of 0.5 nmols IRDye800CW-Fc. Xenografts were collected and a single cell suspension was made by shearing tissue with needles in RPMI media. Bone marrow was flushed from mouse bones with RPMI media. Xenograft and bone marrow cell suspensions were passed through a cell strainer and washed and suspended in a solution of PBS, 0.5% FBS, and 1 mM EDTA. Cells were stained with biotinylated mouse lineage antibodies (Miltenyi Biotec) and streptavidin-PE secondary antibody or secondary alone and analyzed on a Beckman Coulter Gallios flow cytometer. The data was analyzed using FlowJo version 10.5.3.

### Results

### Protein Expression and Labeling

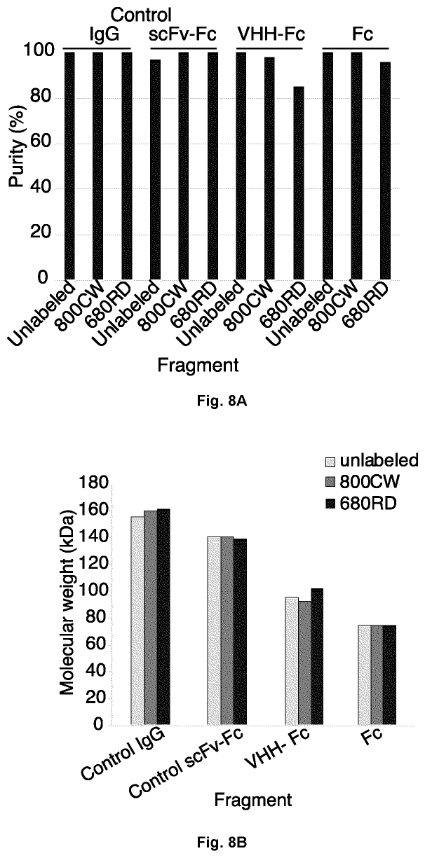

Four IgG1 Fc domain containing proteins (FIG. 1), IgG, scFv-Fc, VHH-Fc, and Fc domain, were expressed and purified. These proteins were conjugated with NHS-IRDye800CW, resulting in a labeling ratio between 1-2. Proteins used for flow cytometry were labeled with IRDye680RD, resulting in a labeling ratio between 1-2. Labeled proteins were analyzed for size and purity using microcapillary electrophoresis (FIG. 8A-B). All proteins were within the expected size and were >85% pure.

Accumulation of VHH-Fc and scFv-Fc Proteins in FaDu Xenografts

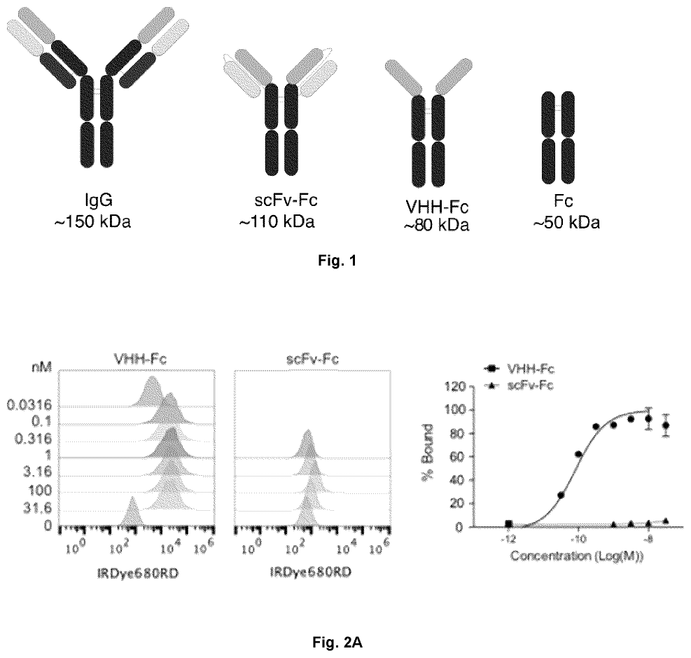

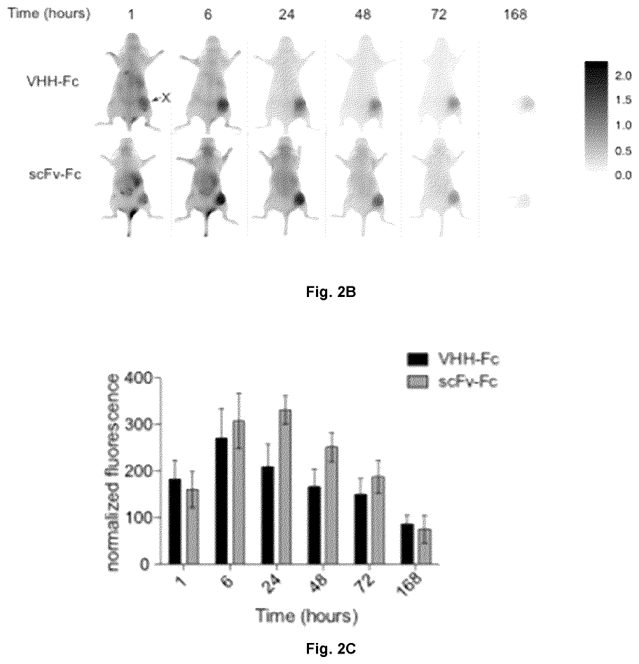

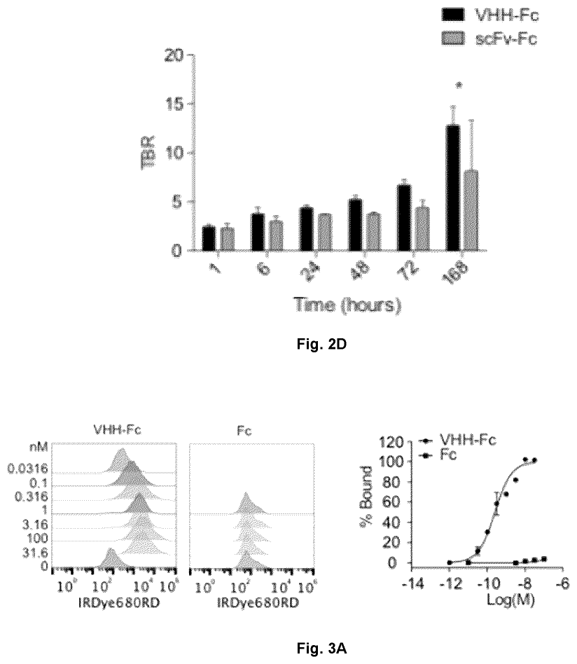

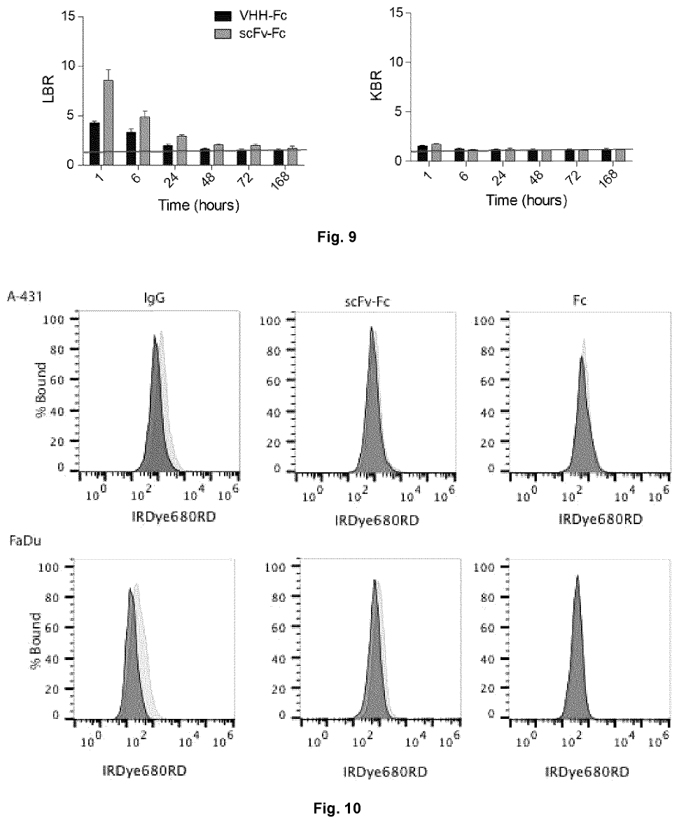

A VHH-Fc fragment that binds to and shows saturation with the FaDu cell line when analyzed by flow cytometry was used (FIG. 2A). An scFv-Fc was used that does not bind FaDu cells in vitro (FIG. 2A). The IRDye800CW-VHH-Fc and IRDye800CW-scFv-Fc were injected into CD-1 nude mice bearing FaDu xenografts and imaged over time. Surprisingly, the IRDye800CW-scFv-Fc accumulated in the FaDu xenograft at slightly higher levels and peaked at a longer time point relative to the IRDye800CW-VHH-Fc, however there was no statistical difference in accumulation in the tumor between the two imaging probes (FIG. 2B-C). Accumulation of the IRDye800CW-VHH-Fc peaked at 6 hours post injection (hpi) with a normalized mean fluorescence intensity (MFI) of 270±64 and the IRDye800CW-scFv-Fc peaked at 24 hpi with a normalized MFI value of 330±30 (FIG. 2C). The liver-to-background ratio (LBR) and kidney-to-background ratio (KBR) were quantitated. There was no significant difference in the tumor-to-background ratio (TBR) between the IRDye800CW-VHH-Fc and the IRDye800CW-scFv-Fc, except at 168 hours (p value <0.05) where the TBR for IRDye800CW-VHH-Fc was slightly higher, however the signal was low (MFI<90) for both imaging probes (FIG. 2D). Both imaging probes cleared through the liver (FIG. 9) and there was no accumulation in the kidney for either imaging probe (FIG. 9).

### Fc Domain Blocks Accumulation of the VHH-Fc in FaDu Xenografts

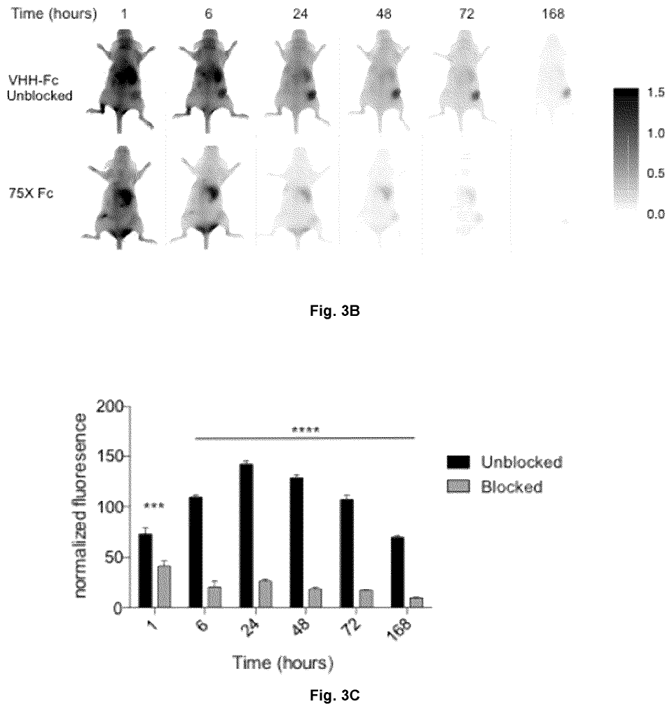

Since both the FaDu binding (VHH-Fc) and non-binding (scFv-Fc) imaging probes accumulated in FaDu xenografts, it was determined if the Fc domain was responsible for the xenograft accumulation. A VHH-Fc blocking experiment using an unlabeled Fc domain was performed. Since the VHH-Fc also binds MDA-MB-231 cells (FIG. 3A), this cell line was used to rule out artifacts that could be attributed to the FaDu cell-line. The IRDye680RD-VHH-Fc and IRDye680RD-Fc domain were tested for binding to MDA-MB-231 cells using flow cytometry (FIG. 3A). The IRDye680RD-VHH-Fc bound and saturated on MDA-MB-231 cells, whereas the IRDye680RD-Fc domain did not bind to MDA-MB-231 cells. The targeted IRDye800CW-VHH-Fc was tested for its ability to accumulate in mice bearing MDA-MB-231 xenografts over time (FIG. 3B). IRDye800CW-VHH-Fc fluorescence signal peaked at 24 hours in the MDA-MB-231 xenograft with an MFI of 140±6 (FIG. 3B-C). However, when a 75-fold excess of the amount of Fc domain was injected prior to injection of IRDye800CW-VHH-Fc, accumulation in the xenograft was significantly blocked (MFI<45 at all time points).

### Variation in Imaging Between Fc Domain Imaging Probes and Xenografts

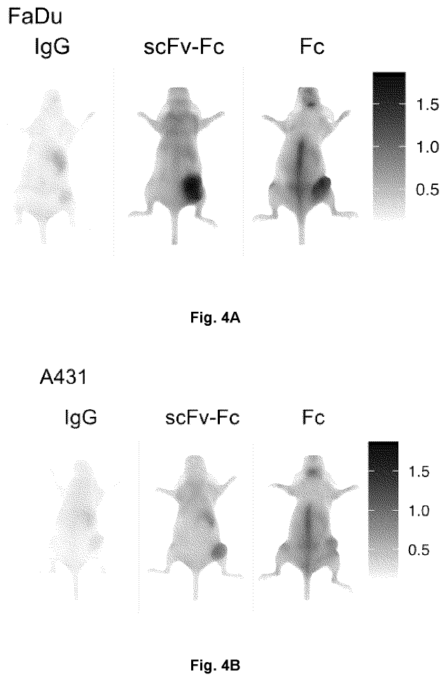

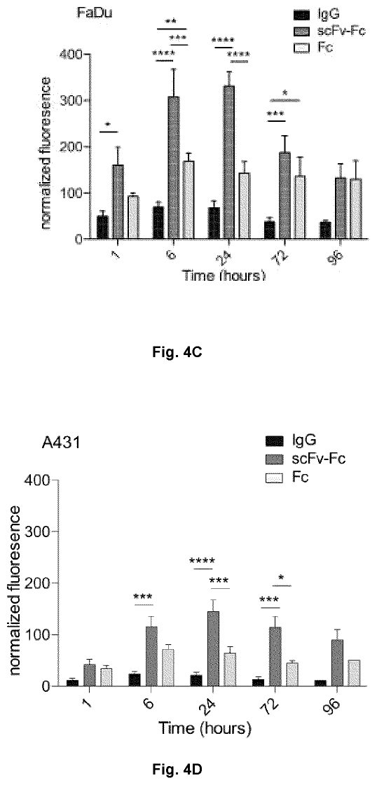

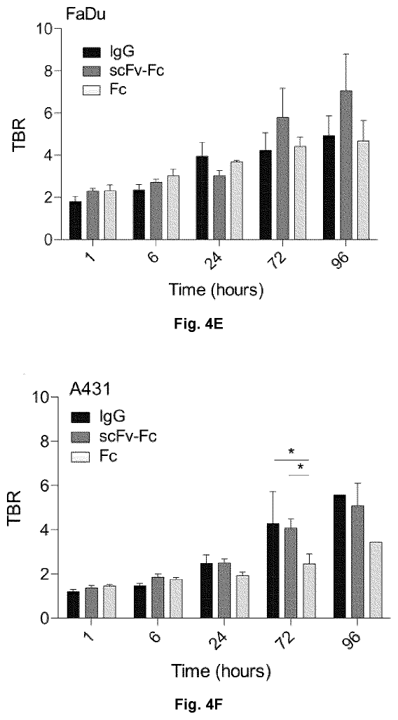

Accumulation of Fc domain containing imaging probes of different sizes in FaDu and A-431 xenografts were compared. Flow cytometry was used to compare binding of IRDye680RD labeled Fc, scFv-Fc, and IgG proteins on FaDu and A-431 cells. None of these imaging probes bound to FaDu and A-431 cell lines in vitro (FIG. 10). Control IgG, scFv-Fc and hFc1 were mixed at 100 nM with FaDu or A-431 cells and were analyzed by flow cytometry. The accumulation of these IRDye800CW labeled imaging probes was measured in FaDu and A-431 xenografts in vivo (FIG. 4A-F). Accumulation of the smaller IRDye800CW-Fc and IRDye800CW-scFv-Fc in the xenografts were higher than the IRDye800CW-IgG. In FaDu xenografts, the IRDye800CW-scFv-Fc accumulated at significantly higher levels than the IRDye800CW-IgG at all time points (p-value≤0.05 at 1 hpi, p-value≤0.0001 at 6 and 24 hpi and p-value≤0.001 at 72 hpi), except at 96 hpi and significantly higher than the IRDye800CW-Fc at 6 and 24 hpi (p-value≤0.001 and 0.0001, respectively) (FIG. 4C). Accumulation of the IRDye800CW-Fc in FaDu xenografts was significantly higher than the control IRDye800CW-IgG at 6 and 72 hpi (p-value≤0.01 and 0.05, respectively) (FIG. 4C). The IRDye800CW-Fc peaked at 6 hours in FaDu xenografts with an MFI of 170±18, whereas the IRDye800CW-scFv-Fc peaked at 24 hours with an MFI of 330±30. The IRDye800CW-IgG had little accumulation in both xenograft types at all time points (MFI<70) (FIG. 4A-B).

In the A-431 xenograft, the IRDye800CW-scFv-Fc accumulated at significantly higher levels than the IRDye800CW-IgG at 6, 24, and 72 hpi (p-value≤0.001 at 6 and 72 hpi and p-value≤0.0001 at 24 hpi) (FIG. 4D) and significantly more than the IRDye800CW-Fc at 24 and 72 hpi (p-value≤0.001 and 0.05, respectively). The IRDye800CW-Fc did not accumulate at high levels in A-431 cells (MFI<72 at all time points). The IRDye800CW-scFv-Fc accumulation in A-431 cells peaked at 24 hours with an MFI of 145±22.

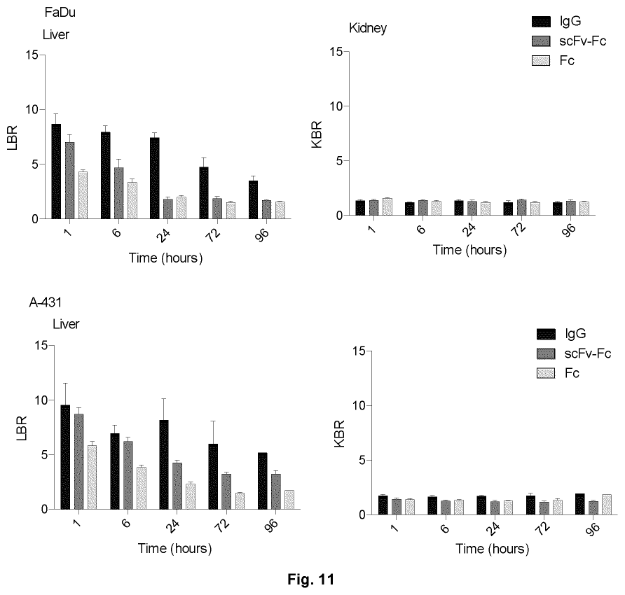

The TBR for the constructs were all similar at each time point in both cell lines (FIG. 4E-F). They all had a TBR around 2 at 1 hour and slowly increased to between 4.5-7 at 96 hours. The TBR for A-431 cells was similar, however both the IgG and the scFv-Fc were significantly higher at 72 hpi compared to the Fc (p-value≤0.05). CD-1 nude mice were injected with targeted control IgG, scFv-Fc or hFc1 and imaged over time and the liver-to-background ratio (LBR) and kidney-to-background ratio (KBR) were quantitated. All three constructs cleared through the liver and had no accumulation in the kidney (FIG. 11).

### Analysis of Fc Binding in Xenografts and Bone Marrow

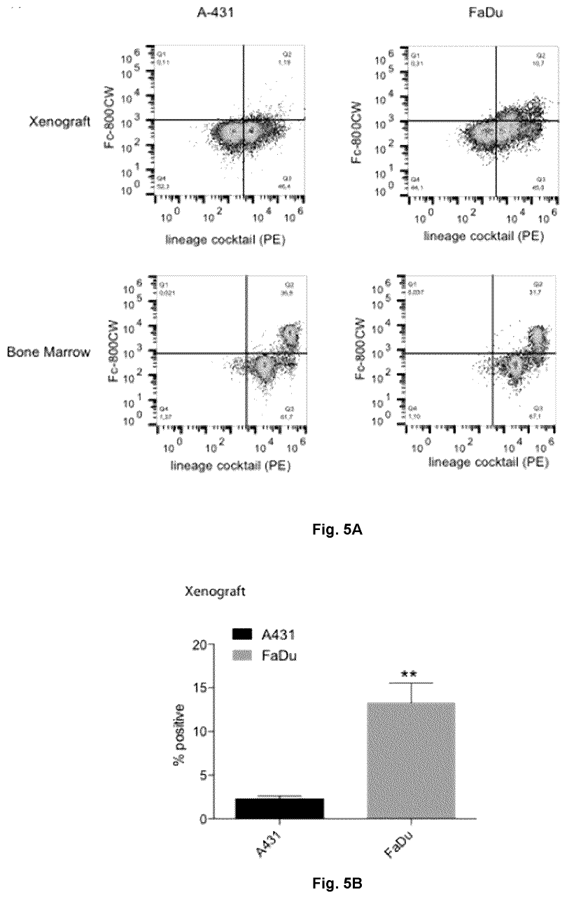

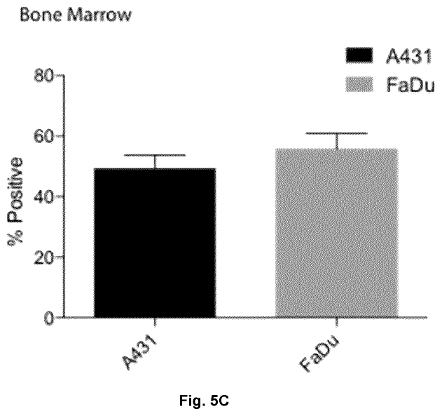

Since injecting an excess of Fc blocked VHH-Fc binding to MDA-MB-231 cells, it was reasoned that the Fc could be binding to Fc receptors on cells that are infiltrating the tumor. To determine if the IRDye800CW-Fc imaging probe was binding tumor-associated immune cells, the binding of the IRDye800CW-Fc to xenograft cells and bone marrow using flow cytometry was analyzed. Xenografts from mice engrafted with A-431 and FaDu cells that had been injected with IRDye800CW-Fc were collected and analyzed by flow cytometry (FIG. 5A-C). Cells collected from xenografts were stained with a lineage marker for differentiated mouse blood cells, which stained approximately half of the cells collected (FIG. 5A). There was <0.5% IRDye800CW positive cells in the lineage negative gate, the majority of IRDye800CW positive cells were found in the lineage marker positive gate, indicating that they were not binding tumor cells, but instead to mouse blood cells. In A-431 xenografts, there was very minimal staining (2.3±0.3%) for the IRDye800CW-Fc (FIG. 5B). Cells from the FaDu xenografts showed significantly more (13.3±2.2%, p=0.01) lineage positive cells and cells were also more positive for the IRDye800CW-Fc than the A-431 xenograft cells. This correlated with the results observed in imaging experiments, where FaDu xenografts were more positive for imaging probe accumulation then A431 xenografts. Bone marrow cells taken from mice showed a similar staining pattern between mice with A-431 and FaDu xenografts with regard to the lineage marker staining (FIG. 5A). In this case almost all the bone marrow cells were lineage marker positive (>98%, independent of the xenograft and 30-40% of these cells were also IRDye800CW-Fc positive) (FIG. 5C). This data showed that the IRDye800CW-Fc was binding to cells in the bone marrow.

### Binding of Fc to Human Peripheral Blood Cells

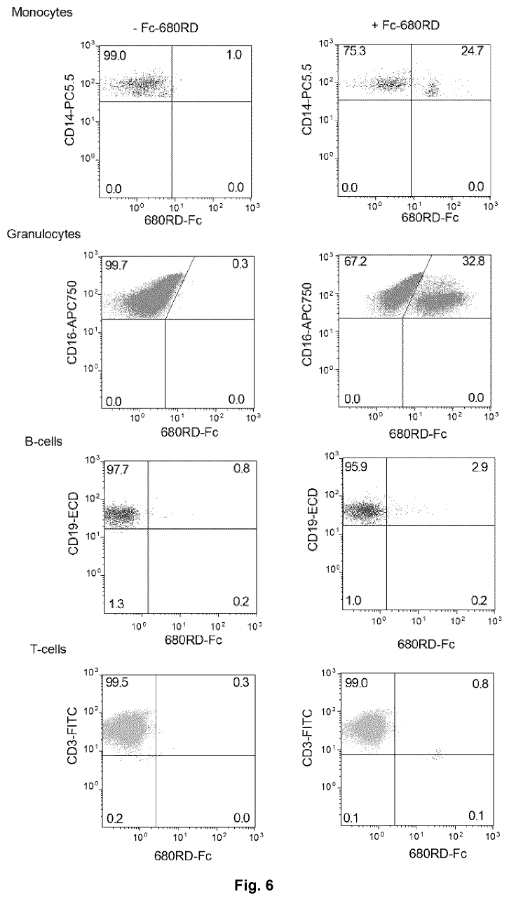

To identify blood cells that interact with the Fc domain imaging probes, the Fc domain was labeled with IRDye680RD and cell types that it interacted with in human peripheral blood were determined using flow cytometry. An immune panel was used, which identifies immune cells based on surface markers to determine the cell types that the IRDye680RD-Fc interacted with (FIG. 6). The IRDye680RD-Fc did not bind to B-cells or T-cells (FIG. 6), however, 25% of monocytes bound and 33% of granulocytes bound to the IRDye680RD-Fc (FIG. 6).

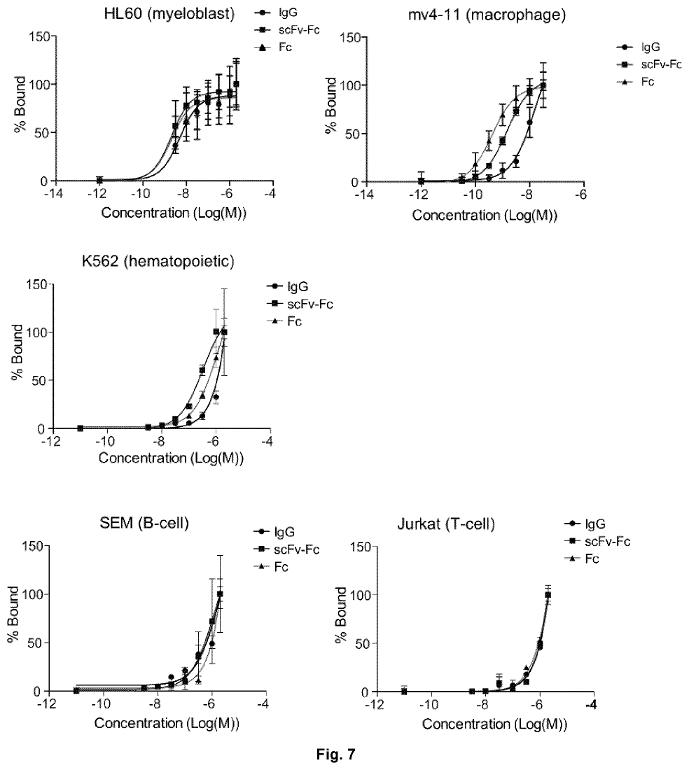

To determine a KD of the IgG, scFv-Fc, and Fc imaging probes with different immune cells, cell lines representative of the different cell types were used. The Fc domain was titrated with different cell lines to obtain binding constants. The Fc containing proteins bound cell lines that represented the same cell types that were interacted with in the human peripheral blood samples. Little binding was observed in the T-cell line (Jurkat) and a B-cell line (SEM). A myeloid cell line (K652) had some weak binding but did not saturate (FIG. 7). Mv4-11 and HL-60 cell lines, which represent macrophages (monocytes) and that can spontaneously differentiate to neutrophils (granulocytes) or monocytes [20,21], respectively bound to the Fc containing proteins with high affinity (FIG. 7). They bound to the HL-60 with binding constants of: IgG: 5±0.3 nM, scFv-Fc: 2.1±0.1 nM, and Fc: 3.6±2.3 nM. They bound to mv4-11 with binding constants of: IgG: 1.7±0.2 nM, scFv-Fc: 1.5±0.05, and Fc: 0.4±0.02 nM.

### DISCUSSION

The larger size of the scFv-Fc may play a role by increasing circulation time, which leads to a longer half-life, giving it more time to accumulate [22]. Interestingly, the IgG showed lower levels of accumulation in the xenograft despite being the largest molecule. This may be due to the IgG having difficulty penetrating the tumor/tumor vasculature [22], or steric hinderance in FcR binding, which is supported by the IgG typically binding the weakest to the cell-lines tested.

When using imaging probes that contain Fc domains it is important to consider that these imaging probes can bind their intended target as well as immune cells, which can cause accumulation in tissues where they may not be expected. This can be exploited to enable an imaging probe to accumulate at higher levels in tissues that contain target and immune cells. The role of the Fc domain in the imaging probe accumulation can be tested by including an unlabeled Fc domain blocking control in imaging experiments. In addition to tumor uptake, accumulation of the Fc domain imaging probe in the backbone, under the armpits, and in the neck and leg bones of mice was observed, which are locations of lymph nodes and large bones containing bone marrow. Lymph nodes and bone marrow contain immune cells and hemopoietic cells, both of which express Fc receptors. Accumulation of the scFv-Fc and IgG were not visible in these areas. Using human PBMCs, it was shown that the Fc domain can bind to granulocytes and monocytes. This is consistent with the literature which shows that certain granulocytes and monocytes have FcγRI (CD64), FcγRII (CD32), and FcγRII (CD16), all of which bind to IgG [23].

It is well established that certain blood cells contain Fc receptors and that they are difficult to analyze by flow cytometry and immuno-histochemistry due to non-specific antibody binding. Often an Fc blocking buffer is used to block these interactions. (Fab) 2 or other non-Fc containing antibody fragments have been developed to avoid Fc binding. Here, how the Fc binding can be exploited for use as an imaging agent to non-invasively image innate immune responses and tumor infiltration by immune cells is shown.

Currently, many cancer diagnostic assays and therapeutic monitoring occurs with the use of biopsies and histology. There is potential to use imaging probes to analyze the tumor microenvironment using targeted molecular imaging. In solid tumors for example, immune cells are involved in tumor infiltration, including T and B lymphocytes, natural killer cells, NK-T cells, dendritic cells, macrophages, neutrophils, eosinophils, and mast cells [24]. Immune cell infiltration of cells varies between different cancers and can potentially be used as a predictor of disease and therapeutic outcome. Neutrophils and macrophages are associated with poor prognosis due to their tumor promoting activities, both of which the Fc binds. Most tumors have immunosuppressive macrophages, which contribute to tumor progression [25]. An increased number of neutrophils in tumors (including but not limited to hepatocellular, esophageal squamous cell carcinoma, melanoma, colorectal cancer, pulmonary adenocarcinoma, and renal cell carcinoma) is also correlated with poor prognosis [4, 5, 26-29]. Imaging probes such as the Fc domain could be used to identify levels of neutrophils and macrophages. Non-invasive imaging can be used to determine levels of innate immune infiltration and potentially aid in identifying disease states. The benefit of monitoring innate immune response extends beyond cancer and would be useful for other diseases such as cardiovascular diseases, inflammation, ischemia, and multiple sclerosis [30, 31]. By labeling the Fc with a fluorescent, microbubble, or radioactive moiety, the probe could be imaged with a fluorescent endoscope, ultrasound or with PET, respectively, to identify areas of inflammation.

Monitoring and predicting therapeutic efficacy are important areas that would benefit from non-invasive imaging tools. Most therapies are expensive and prone to side effects, which can lead to non-compliance. Predicting efficacies of therapies and monitoring efficacy would avoid therapy costs, unnecessary treatments, and side effects. For example, neutrophil infiltration in metastatic colorectal cancer not only indicates lower survival rates but also shows negative effects against bevacizumab treatment [5]. Knowing this would prevent these patients from adverse events associated with bevacizumab treatments. Macrophages have also been tracked to monitor the inhibitory effect of dexamethasone to macrophage migration [32], which could be useful in monitoring recruitment or disappearance of macrophages before and during other therapies.

The Fc imaging probe will be useful for determining if immune cell infiltration is present in the tumor environment. While establishing xenografts in athymic CD-1 nude mice, which lack T-cells, it was observed that some cell-lines are more difficult to engraft. For example, FaDu cells have a lower rate of engraftment versus A-431 cells. Xenograft rejection has been previously shown to be due to infiltrating macrophages and T-cells [33]. The lower rate of FaDu engraftment correlates with the Fc imaging probe uptake in the graft and could be used to monitor immune reactions in therapeutic animal models. The Fc imaging probe is also useful to study other types of inflammatory diseases.

### REFERENCES

1. Karin M, Lawrence T, Nizet V. Innate immunity gone awry: Linking microbial infections to chronic inflammation and cancer. Cell. 2006; 124:823-35.
2. Vogelstein B, Kinzler K W. Cancer genes and the pathways they control. Nat Med. 2004; 10:789-99.
3. Jeong J, Suh Y, Jung K. Context drives diversification of monocytes and neutrophils in orchestrating the tumor microenvironment. Front Immunol. 2019; 10:1817.
4. Jensen H K, Donskov F, Marcussen N, Nordsmark M, Lundbeck F, von der Maase H. Presence of intratumoral neutrophils is an independent prognostic factor in localized renal cell carcinoma. J Clin Oncol. 2009; 27:4709-17.
5. Schiffmann L M, Fritsch M, Gebauer F, Günther S D, Stair N R, Seeger J M, et al. Tumour-infiltrating neutrophils counteract anti-vegf therapy in metastatic colorectal cancer. Br J Cancer. 2018; 120:69-78.
6. Zhuang H, Pourdehnad M, Lambright E S, Yamamoto A J, Lanuti M, Li P, et al. Dual time point 18F-FDG PET imaging for differentiating malignant from inflammatory processes. J Nucl Med. 2001; 42:1412-7.
7. McCracken M N, Radu C G. Targeted non-invasive imaging of the innate immune response. Proc Natl Acad Sci USA. 2015; 112:5868-9.
8. Eisenblätter M, Ehrchen J, Varga G, Sunderkötter C, Heindel W, Roth J, et al. In vivo optical imaging of cellular inflammatory response in granuloma formation using fluorescence-labeled macrophages. J Nucl Med. 2009; 50:1676-82.
9. Lee H W, Jeon Y H, Hwang M H, Kim J E, Park T I, Ha J H, et al. Dual reporter gene imaging for tracking macrophage migration using the human sodium iodide symporter and an enhanced firefly luciferase in a murine inflammation model. Mol Imaging Biol. 2013; 15:703-12.
10. Distribution studies of 111 in-oxine-labeled peritoneal mononuclear cells in tumor-bearing rats. Int J of Nucl Med Biol. 1985; 12:53-5.
11. Nahrendorf M, Zhang H, Hembrador S, Panizzi P, Sosnovik D E, Aikawa E, et al. Nanoparticle PET-CT imaging of macrophages in inflammatory atherosclerosis. Circulation. 2008; 117:379-87.
12. Lee H W, Gangadaran P, Kalimuthu S, Ahn B C. Advances in molecular imaging strategies for in vivotracking of immune cells. Biomed Res Int. 2016; 2016:1946585.
13. Nair-Gill E, Wiltzius S M, Wei X X, Cheng D, Riedinger M, Radu C G, Witte O N. PET probes for distinct metabolic pathways have different cell specificities during immune responses in mice. J Clin Invest. 2010; 120:2005-15.
14. Radu C G, Shu C J, Shelly S M, Phelps M E, Witte O N. Positron emission tomography with computed tomography imaging of neuroinflammation in experimental autoimmune encephalomyelitis. Proc Natl Acad Sci USA. 2007; 104:1937-42.
15. Eshghi N, Lundeen T F, Kuo P H. Dynamic adaptation of tumor immune response with nivolumab demonstrated by 18F-FDG PET/C T. Clin Nucl Med. 2018; 43:114-6.
16. Lugade A A, Moran J P, Gerber S A, Rose R C, Frelinger J G, Lord E M. Local radiation therapy of B16 melanoma tumors increases the generation of tumor antigen-specific effector cells that traffic to the tumor. J Immunol. 2005; 174:7516-23.
17. van Elsas A, Hurwitz A A, Allison J P. Combination immunotherapy of B16 melanoma using anti-cytotoxic T lymphocyte-associated antigen 4 (ctla-4) and granulocyte/macrophage colony-stimulating factor (gm-csf)-producing vaccines induces rejection of subcutaneous and metastatic tumors accompanied by autoimmune depigmentation. J Exp Med. 1999; 190:355-66.
18. Bernhard W, El-Sayed A, Barreto K, Gonzalez C, Hill W, Parada A C, et al. Near infrared fluorescence imaging of EGFR expression in vivo using irdye800 cw-nimotuzumab. Oncotarget. 2018; 9:6213-27.
19. Ayman El-Sayed, Wendy Bernhard, Kris Barreto, Carolina Gonzalez, Wayne Hill, Landon Pastushok, Humphrey Fonge, C. Ronald Geyer. Evaluation of antibody fragment properties for near-infrared fluorescence imaging of her3-positive cancer xenografts. Theranostics. 2018; 8:4856-69.
20. Gallagher R, Collins S, Trujillo J, McCredie K, Ahern M, Tsai S, et al. Characterization of the continuous, differentiating myeloid cell line (HL-60) from a patient with acute promyelocytic leukemia. Blood. 1979; 54:3.
21. Breitman T R, Selonick S E, Collins S J. Induction of Differentiation of Human Promyelocytic Leukemia Cell Line (HL-60) by Retinoic Acid. Proc Natl Acad Sci USA. 1980; 77:2936-40.
22. Xenaki K T, Oliveira S, van Bergen En Henegouwen P M P. Antibody or antibody fragments: Implications for molecular imaging and targeted therapy of solid tumors. Front Immunol. 2017; 8:1287.
23. Woof J M, van Egmond M, Kerr M A. Chapter 13: Fc Receptors. In: Mestecky J, Lamm M E, Ogra P, Strober W, Bienenstock, McGhee J, Mayer L, Ed. Mucosal Immunology. 3^{rd} ed. Cambridge USA: Academic Press; 2005:251-65.
24. Pagès F, Galon J, Dieu-Nosjean M-C, Tartour E, Sautès-Fridman C, Fridman W H. Immune infiltration in human tumors: A prognostic factor that should not be ignored. Oncogene. 2009; 29:1093-102.
25. Mantovani A, Sica A, Sozzani S, Allavena P, Vecchi A, Locati M. The chemokine system in diverse forms of macrophage activation and polarization. Trends Immunol. 2004; 25:677-86.
26. He G, Zhang H, Zhou J, Wang B, Chen Y, Kong Y, et al. Peritumoral neutrophils negatively regulate adaptive immunity via the PD-L1/PD-1 signalling pathway in hepatocellular carcinoma. J Exp Clin Cancer Res. 2015; 34:1-11.
27. Wang J, Jia Y, Wang N, Zhang X, Tan B, Zhang G, Cheng Y. The clinical significance of tumor-infiltrating neutrophils and neutrophil-to-cd8+ lymphocyte ratio in patients with resectable esophageal squamous cell carcinoma. J Transl Med. 2014; 12:1-10.
28. Jensen T O, Schmidt H, Møller H J, Donskov F, Høyer M, Sjoegren P, et al. Intratumoral neutrophils and plasmacytoid dendritic cells indicate poor prognosis and are associated with pstat3 expression in AJCC stage I/II melanoma. Cancer. 2012; 118:2476-85.
29. Wislez M, Rabbe N, Marchal J, Milleron B, Crestani B, Mayaud C, et al. Hepatocyte Growth Factor Production by Neutrophils Infiltrating Bronchioloalveolar Subtype Pulmonary Adenocarcinoma: Role in Tumor Progression and Death. Cancer Res. 2003; 63:1405-12.
30. Seth A, Park H S, Hong K S. Current perspective on in vivo molecular imaging of immune cells. Molecules. 2017; 22.
31. Kirschbaum K, Sonner J K, Zeller M W, Deumelandt K, Bode J, Sharma R, et al. In vivo nanoparticle imaging of innate immune cells can serve as a marker of disease severity in a model of multiple sclerosis. Proc Natl Acad Sci USA. 2016; 113:13227-32.
32. Kang S, Lee H W, Jeon Y H, Singh T D, Choi Y J, Park J Y, et al. Combined fluorescence and magnetic resonance imaging of primary macrophage migration to sites of acute inflammation using near-infrared fluorescent magnetic nanoparticles. Mol Imaging and Biol. 2015; 17:643-51.
33. Fox A, Mountford J, Braakhuis A, Harrison L C. Innate and adaptive immune responses to nonvascular xenografts: Evidence that macrophages are direct effectors of xenograft rejection. J Immunol. 2001; 166:2133-40.

1. A method of detecting the presence of tumor infiltrating Fc receptor-expressing immune cells in a mammalian subject afflicted with a cancer, the method comprising:
a. administering a non-specific imaging probe comprising one or more Fc domain(s) labeled with a detectable moiety to the subject;
b. subjecting the subject to imaging; and
c. identifying one or more tumor(s) comprising infiltrating Fc receptor-expressing immune cells,
wherein detection of Fc receptor-expressing immune cells is indicative of the presence of infiltrating immune cells in the one or more tumor(s).

2. A method of identifying a mammalian subject afflicted with a cancer as having a poor prognosis, the method comprising:
a. detecting the presence of tumor infiltrating Fc receptor-expressing immune cells in the subject using the method of claim 1; and
b. quantifying the level of neutrophils and/or macrophages in one or more tumor(s);
c. quantifying the level of neutrophils and/or macrophages in a normal surrounding tissue sample; and
d. comparing the level of neutrophils and/or macrophages in the one or more tumor(s) with the normal surrounding tissue sample,
wherein an increased level of neutrophils and/or macrophages in the one or more tumor(s) as compared to the level of neutrophils and/or macrophages in the normal surrounding tissue sample is indicative of a poor prognosis.

3. The method of claim 2, wherein the type of immune cells detected are identified by isolating the one or more tumor(s) or a biopsy thereof and subjecting the one or more tumor(s) or biopsy to flow cytometry.

4. The method of claim 1, wherein the cancer is breast cancer, hepatocellular carcinoma, squamous cell carcinoma, esophageal squamous cell carcinoma, melanoma, colorectal cancer, pulmonary adenocarcinoma, or renal cell carcinoma.

5. The method of claim 1, wherein the cancer is breast cancer.

6. The method of claim 1, wherein the cancer is squamous cell carcinoma.

7. The method of claim 1, wherein the non-specific imaging probe is administered to the subject prior to receiving a cancer treatment and/or after receiving a cancer treatment.

8. (canceled)

9. (canceled)

10. A method of detecting the presence of one or more site(s) of inflammation characterized by Fc receptor-expressing immune cells in a mammalian subject afflicted with an inflammatory disease or disorder, the method comprising:
a. administering a non-specific imaging probe comprising one or more Fc domain(s) labeled with a detectable moiety to the subject;
b. subjecting the subject to imaging; and
c. identifying one or more site(s) of inflammation,
wherein detection of an accumulation of Fc receptor-expressing immune cells is indicative of the presence of one or more site(s) of inflammation.

11. The method of claim 10, wherein the inflammatory disease or disorder is Crohn's disease, atherosclerosis, inflammatory bowel disorder, arthritis, colitis, type II diabetes or psoriasis.

12. The method of claim 10, wherein the non-specific imaging probe is administered to the subject prior to receiving a treatment for an inflammatory disease or disorder and/or after receiving a treatment for an inflammatory disease or disorder.

13. (canceled)

14. (canceled)

15. The method of claim 1, wherein the one or more Fc domain(s) is/are one or more IgG1 Fc domain(s).

16. The method of claim 1, wherein the one or more Fc domain(s) comprise(s) a signal sequence and CH2 and CH3 domains.

17. The method of claim 1, wherein the one or more Fc domain(s) is/are one or more human Fc domain(s).

18. The method of claim 1, wherein the one or more Fc domain(s) comprise(s) the amino acid sequence set forth in SEQ ID NO: 1.

19. The method of claim 1, wherein the detectable moiety is a fluorescent moiety.

20. The method of claim 1, wherein the detectable moiety is IRDye680RD NHS ester or IRDye800CW NHS ester.

21. The method of claim 1, wherein the non-specific imaging probe comprises one Fc domain.

22. The method of claim 1, wherein the subject is a human.

23. The method of claim 1, wherein the non-specific imaging probe is formulated for injection.

24. The method of claim 1, wherein the non-specific imaging probe is administered to the subject more than once at different time periods.

25.-47. (canceled)
